(19) United States
(12) Patent Application Publication
(10) Pub. No.: US 2020/0261463 A1
(43) Pub. Date: Aug. 20, 2020

(54) TREATMENT OF CANCER USING ARYL DIHYDRO-2H-BENZO[B][1,4]OXAZINE SULFONAMIDE COMPOUNDS

(71) Applicant: Lycera Corporation, Ann Arbor, MI (US)

(72) Inventors: Laura Lee Celeste, Ann Arbor, MI (US); Xiao Hu, Northville, MI (US); H. Jeffrey Wilkins, Sellersville, PA (US)

(21) Appl. No.: 16/797,104

(22) Filed: Feb. 21, 2020

Related U.S. Application Data
(63) Continuation of application No. PCT/US2018/047902, filed on Aug. 24, 2018.
(60) Provisional application No. 62/632,496, filed on Feb. 20, 2018.
(60) Provisional application No. 62/550,011, filed on Aug. 25, 2017.

(51) Int. Cl. A61K 31/538; A61P 35/04

(52) U.S. Cl. CPC A61K 31/538; A61K 9/0053; A61P 35/04

(57) ABSTRACT

The invention provides methods for treating a cancer selected from the group consisting of (i) a squamous cell carcinoma located in a patient's head and/or neck and (ii) a urothelial carcinoma using aryl dihydro-2H-benzo[b][1,4]oxazine sulfonamide compounds.

### CROSS-REFERENCE TO RELATED APPLICATIONS

This application is a continuation of International (PCT) Patent Application Serial No. PCT/US2018/047902, filed Aug. 24, 2018, which claims the benefit of and priority to U.S. Provisional Patent Application Ser. No. 62/632,496, filed Feb. 20, 2018, and U.S. Provisional Patent Application Ser. No. 62/550,011, filed Aug. 25, 2017; the contents of each of which are hereby incorporated by reference in their entirety.

### FIELD OF THE INVENTION

The invention provides methods for treating a cancer selected from the group consisting of (i) a squamous cell carcinoma located in a patient's head and/or neck and (ii) a urothelial carcinoma using aryl dihydro-2H-benzo[b][1,4]oxazine sulfonamide compounds.

### BACKGROUND

Retinoid-related orphan receptors (ROR) are reported to have an important role in numerous biological processes. See, for example, Dussault et al. in Mech. Dev. (1998) vol. 70, 147-153; and Andre et al. in EMBO J. (1998) vol. 17, 3867-3877. Scientific investigations relating to each of retinoid-related orphan receptors RORα, RORβ, and RORγ have been described in the literature. See, for example, Hirose et al. in Biochem. Biophys. Res. Commun. (1994) vol. 205, 1976-1983; Giguere et al. in Genes. Dev. (1994) vol. 8, 538-553; Medvedev et al. in Gene (1996) vol. 181, 199-206; Ortiz et al. in Mol. Endocrinol. (1995) vol. 9, 1679-1691; and A. M. Jetten in Curr Drug Targets Inflamm Allergy (2004) vol. 3, 395-412). Continuing research in this field is spurred by the promise of developing new therapeutic agents to treat medical disorders associated with retinoid-related orphan receptor activity.

RORγ has been reported to be expressed in high concentration in various tissues, such as thymus, kidney, liver, muscle, and certain fat tissue. See, for example, Hirose et al. in Biochem. Biophys. Res. Commun. (1994) vol. 205, 1976-1983; Medvedev et al. in Gene (1996) vol. 181, 199-206; Ortiz et al. in Mol. Endocrinol. (1995) vol. 9, 1679-1691; and He et al. in Immunity (1998) vol. 9, 797-806. Two isoforms of RORγ have been identified and are referred to as γ1 and γ2 (also referred to as RORγt). See, for example, He et al. in Immunity (1998) vol. 9, 797-806. Expression of the γ2 isoform has been reported to appear in, for example, double-positive thymocytes. See, for example, He et al. in Immunity (1998) vol. 9, 797-806; and Villey et al. in Eur. J. Immunol. (1999) vol. 29, 4072-4080. RORγt plays a critical role in regulating differentiation of Th17 cells, a subset of T helper lymphocytes. See, for example, Ivanov et al. in Cell (2006) vol. 126, 1121-1133. Th17 cells are important for recruiting tumor-killing cytotoxic CD8+ T cells and natural killer cells into the tumor microenvironment. The level of Th17 cells correlated positively with patient survival or slower disease progression in certain cancers. See, for example, Kryczek et al. in Blood (2009) vol 114, 1141-1149; and Sfanos et al. in Clinical Cancer Research (2008) vol 14, 3254-3261. Compounds capable of enhancing RORγt activity are thus contemplated to provide a therapeutic benefit in the treatment of cancer.

Cancer continues to be a significant health problem despite the substantial research efforts and scientific advances reported in the literature for treating this disease. Head and neck cancers impact a significant number of patients, and current treatment options are insufficient for all patients. Urothelial carcinoma is another cancer for which better treatment options are needed.

Accordingly, a need exists for improved treatments for cancer of the head and neck and urothelial carcinomas. The present invention addresses this need and provides other related advantages.

### SUMMARY

The invention provides methods for treating a cancer selected from the group consisting of (i) a squamous cell carcinoma located in a patient's head and/or neck and (ii) a urothelial carcinoma using aryl dihydro-2H-benzo[b][1,4]oxazine sulfonamide compounds. The therapeutic method desirably involves oral administration of an aryl dihydro-2H-benzo[b][1,4]oxazine sulfonamide, which has RORγ agonist activity. Various aspects and embodiments of the invention are described in further detail below.

Accordingly, one aspect of the invention provides a method of treating a cancer selected from the group consisting of (i) a squamous cell carcinoma located in a patient's head and/or neck and (ii) a urothelial carcinoma. The method comprises administering to a patient in need thereof a therapeutically effective amount of a compound of Formula I, to thereby treat the cancer, wherein Formula I is represented by:

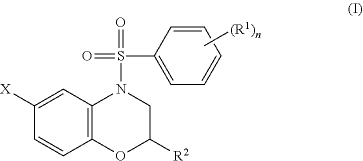

or a pharmaceutically acceptable salt thereof; wherein X is phenyl substituted by 1, 2, or 3 substituents independently selected from the group consisting of C₁₋₂ fluoroalkoxy, C₁₋₂ alkoxy, chloro, fluoro, and C₁₋₂ fluoroalkyl; R¹ represents independently for each occurrence C₁₋₂ fluoroalkyl, chloro, fluoro, cyclopropyl, or C₁₋₃ alkyl; R² is —(C₁₋₆ alkylene)-CO₂H; and n is 1 or 2.

Another aspect of the invention provides a method of treating a cancer that is a squamous cell carcinoma located in a patient's head and/or neck. The method comprises administering to a patient in need thereof a therapeutically effective amount of a compound of Formula I, as described herein, to thereby treat the cancer.

Another aspect of the invention provides a method of treating a cancer that is a urothelial carcinoma. The method comprises administering to a patient in need thereof a therapeutically effective amount of a compound of Formula I, as described herein, to thereby treat the cancer.

Another aspect of the invention provides pharmaceutical compositions for use in the therapeutic methods described herein. Also provided are medical kits containing a compound of Formula I and instructions for treating a cancer selected from the group consisting of (i) a squamous cell carcinoma located in a patient's head and/or neck and (ii) a urothelial carcinoma.

### DETAILED DESCRIPTION

The invention provides methods for treating a cancer selected from the group consisting of (i) a squamous cell carcinoma located in a patient's head and/or neck and (ii) a urothelial carcinoma using aryl dihydro-2H-benzo[b][1,4]oxazine sulfonamide compounds. The therapeutic method desirably involves oral administration of an aryl dihydro-2H-benzo[b][1,4]oxazine sulfonamide, which has RORγ agonist activity. Various aspects of the invention are set forth below in sections; however, aspects of the invention described in one particular section are not to be limited to any particular section. Further, when a variable is not accompanied by a definition, the previous definition of the variable controls.

### Definitions

The terms used herein have their ordinary meaning and the meaning of such terms is independent at each occurrence thereof. That notwithstanding and except where stated otherwise, the following definitions apply throughout the specification and claims. Chemical names, common names, and chemical structures may be used interchangeably to describe the same structure. If a chemical compound is referred to using both a chemical structure and a chemical name, and an ambiguity exists between the structure and the name, the structure predominates. These definitions apply regardless of whether a term is used by itself or in combination with other terms, unless otherwise indicated. Hence, the definition of “alkyl” applies to “alkyl” as well as the “alkyl” portions of “—O-alkyl” etc.

The term “alkyl” refers to a saturated straight or branched hydrocarbon, such as a straight or branched group of 1-12, 1-10, or 1-6 carbon atoms, referred to herein as C₁-C₁₂ alkyl, C₁-C₁₀ alkyl, and C₁-C₆ alkyl, respectively. Exemplary alkyl groups include, but are not limited to, methyl, ethyl, propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, butyl, isobutyl, t-butyl, pentyl, isopentyl, neopentyl, hexyl, heptyl, octyl, etc.

The term “alkylene” refers to a diradical of an alkyl group. Exemplary alkylene groups include —CH₂—, —CH₂CH₂—, and —CH₂C(H)(CH₃)CH₂—.

The term “cycloalkyl” refers to a monovalent saturated cyclic, bicyclic, or bridged cyclic (e.g., adamantyl) hydrocarbon group of 3-12, 3-8, 4-8, or 4-6 carbons, referred to herein, e.g., as “C₃-C₆ cycloalkyl,” derived from a cycloalkane. Exemplary cycloalkyl groups include cyclohexyl, cyclopentyl, cyclobutyl, and cyclopropyl.

The term “haloalkyl” refers to an alkyl group that is substituted with at least one halogen. Exemplary haloalkyl groups include —CH₂F, —CHF₂, —CF₃, —CH₂CF₃, —CF₂CF₃, and the like.

The term “aralkyl” refers to an alkyl group substituted with an aryl group. Exemplary aralkyl groups include

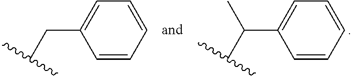

The term “heteroaralkyl” refers to an alkyl group substituted with a heteroaryl group.

The terms “alkenyl” and “alkynyl” are art-recognized and refer to unsaturated aliphatic groups analogous in length and possible substitution to the alkyls described above, but that contain at least one double or triple bond respectively.

The term “aryl” is art-recognized and refers to a carbocyclic aromatic group. Representative aryl groups include phenyl, naphthyl, anthracenyl, and the like. Unless specified otherwise, the aromatic ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “aryl” also includes polycyclic aromatic ring systems having two or more carbocyclic rings in which two or more carbons are common to two adjoining rings (the rings are “fused rings”) wherein all of the fused rings are aromatic rings, e.g., in a naphthyl group.

The term “heteroaryl” is art-recognized and refers to aromatic groups that include at least one ring heteroatom. In certain instances, a heteroaryl group contains 1, 2, 3, or 4 ring heteroatoms (e.g., O, N, and S). Representative examples of heteroaryl groups include pyrrolyl, furanyl, thiophenyl, imidazolyl, oxazolyl, thiazolyl, triazolyl, pyrazolyl, pyridinyl, pyrazinyl, pyridazinyl and pyrimidinyl, and the like. Unless specified otherwise, the heteroaryl ring may be substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. The term “heteroaryl” also includes polycyclic aromatic ring systems having two or more rings in which two or more ring atoms are common to two adjoining rings (the rings are “fused rings”) wherein all of the fused rings are heteroaromatic, e.g., in a naphthyridinyl group. In certain embodiments, the heteroaryl is a 5-6 membered monocyclic ring or a 9-10 membered bicyclic ring.

The terms ortho, meta, and para are art-recognized and refer to 1,2-, 1,3- and 1,4-disubstituted benzenes, respectively. For example, the names 1,2-dimethylbenzene and ortho-dimethylbenzene are synonymous.

As used herein, the terms “heterocyclic” and “heterocyclyl” represent, for example, an aromatic or nonaromatic ring (e.g., a monocyclic or bicyclic ring) containing one or more heteroatoms. The heteroatoms can be the same or different from each other. Examples of heteratoms include, but are not limited to nitrogen, oxygen and sulfur. Aromatic and nonaromatic heterocyclic rings are well-known in the art. Some nonlimiting examples of aromatic heterocyclic rings include, but are not limited to, pyridine, pyrimidine, indole, purine, quinoline and isoquinoline. Nonlimiting examples of nonaromatic heterocyclic compounds include, but are not limited to, piperidine, piperazine, morpholine, pyrrolidine and pyrazolidine. Examples of oxygen containing heterocyclic rings include, but are not limited to, furan, oxirane, 2H-pyran, 4H-pyran, 2H-chromene, benzofuran, and 2,3-dihydrobenzo[b][ 1,4]dioxine. Examples of sulfur-containing heterocyclic rings include, but are not limited to, thiophene, benzothiophene, and parathiazine. Examples of nitrogen containing rings include, but are not limited to, pyrrole, pyrrolidine, pyrazole, pyrazolidine, imidazole, imidazoline, imidazolidine, pyridine, piperidine, pyrazine, piperazine, pyrimidine, indole, purine, benzimidazole, quinoline, isoquinoline, triazole, and triazine. Examples of heterocyclic rings containing two different heteroatoms include, but are not limited to, phenothiazine, morpholine, parathiazine, oxazine, oxazole, thiazine, and thiazole. The heterocyclic ring is optionally further substituted at one or more ring positions with, for example, halogen, azide, alkyl, aralkyl, alkenyl, alkynyl, cycloalkyl, hydroxyl, alkoxyl, amino, nitro, sulfhydryl, imino, amido, carboxylic acid, —C(O)alkyl, —CO₂alkyl, carbonyl, carboxyl, alkylthio, sulfonyl, sulfonamido, sulfonamide, ketone, aldehyde, ester, heterocyclyl, aryl or heteroaryl moieties, —CF₃, —CN, or the like. In certain embodiments, the heterocyclyl group is a 3-7 membered ring that, unless specified otherwise, is substituted or unsubstituted.

The term “heterocycloalkyl” refers to a saturated heterocyclyl group having, for example, 3-7 ring atoms selected from carbon and heteroatoms (e.g., O, N, or S).

The terms “amine” and “amino” are art-recognized and refer to both unsubstituted and substituted amines, e.g., a moiety that may be represented by the general formulas:

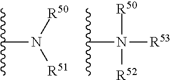

wherein R⁵⁰, R⁵¹, R⁵² and R⁵³ each independently represent a hydrogen, an alkyl, an alkenyl, —(CH₂)ₘ—R⁶¹, or R⁵⁰ and R⁵¹, taken together with the N atom to which they are attached complete a heterocycle having from 4 to 8 atoms in the ring structure; R⁶¹ represents an aryl, a cycloalkyl, a cycloalkenyl, a heterocycle or a polycycle; and m is zero or an integer in the range of 1 to 8. In certain embodiments, only one of R⁵⁰ or R⁵¹ may be a carbonyl, e.g., R⁵⁰, R⁵¹ and the nitrogen together do not form an imide. In other embodiments, R⁵⁰ and R⁵¹ (and optionally R⁵²) each independently represent a hydrogen, an alkyl, an alkenyl, or —(CH₂)ₘ—R⁶¹.

The terms “alkoxyl” or “alkoxy” are art-recognized and refer to an alkyl group, as defined above, having an oxygen radical attached thereto. Representative alkoxyl groups include methoxy, ethoxy, propyloxy, tert-butoxy and the like. An “ether” is two hydrocarbons covalently linked by an oxygen. Accordingly, the substituent of an alkyl that renders that alkyl an ether is or resembles an alkoxyl, such as may be represented by one of —O-alkyl, —O-alkenyl, —O-alkynyl, and —O—(CH₂)ₘ—R⁶¹, where m and R⁶¹ are described above.

The term “oxo” is art-recognized and refers to a “═O” substituent. For example, a cyclopentane substituted with an oxo group is cyclopentanone.

The symbol “ [symbol] ” indicates a point of attachment.

The term “substituted” means that one or more hydrogens on the atoms of the designated group are replaced with a selection from the indicated group, provided that the atoms' normal valencies under the existing circumstances are not exceeded, and that the substitution results in a stable compound. Combinations of substituents and/or variables are permissible only if such combinations result in stable compounds. The terms “stable compound” or “stable structure” refer to a compound that is sufficiently robust to survive isolation to a useful degree of purity from a reaction mixture, and formulation into an efficacious therapeutic agent.

When any substituent or variable occurs more than one time in any constituent or the compound of the invention, its definition on each occurrence is independent of its definition at every other occurrence, unless otherwise indicated.

It should also be noted that any carbon as well as heteroatom with unsatisfied valences in the text, schemes, examples and tables herein is assumed to have the sufficient number of hydrogen atom(s) to satisfy the valences.

One or more compounds of the invention may exist in unsolvated as well as solvated forms with pharmaceutically acceptable solvents such as water, ethanol, and the like, and it is intended that the invention embrace both solvated and unsolvated forms. “Solvate” means a physical association of a compound of this invention with one or more solvent molecules. This physical association involves varying degrees of ionic and covalent bonding, including hydrogen bonding. In certain instances the solvate will be capable of isolation, for example when one or more solvent molecules are incorporated in the crystal lattice of the crystalline solid. “Solvate” encompasses both solution-phase and isolatable solvates. Non-limiting examples of suitable solvates include ethanolates, methanolates, and the like. “Hydrate” is a solvate wherein the solvent molecule is H₂O.

Certain compounds contained in compositions of the present invention may exist in particular geometric or stereoisomeric forms. Further, certain compounds described herein may be optically active. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. The compounds may contain one or more stereogenic centers. For example, asymmetric carbon atoms may be present in a substituent such as an alkyl group. All such isomers, as well as mixtures thereof, are intended to be included in this invention, such as, for example, racemic mixtures, single enantiomers, diastereomeric mixtures and individual diastereomers. Additional asymmetric centers may be present depending upon the nature of the various substituents on the molecule. Each such asymmetric center will independently produce two optical isomers, and it is intended that all of the possible optical isomers, diastereomers in mixtures, and pure or partially purified compounds are included within the ambit of this invention.

Diastereomeric mixtures can be separated into their individual diastereomers on the basis of their physical chemical differences by methods known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Alternatively, a particular enantiomer of a compound of the present invention may be prepared by asymmetric synthesis. Still further, where the molecule contains a basic functional group (such as amino) or an acidic functional group (such as carboxylic acid) diastereomeric salts are formed with an appropriate optically-active acid or base, followed by resolution of the diastereomers thus formed by fractional crystallization or chromatographic means known in the art, and subsequent recovery of the pure enantiomers.

Individual stereoisomers of the compounds of the invention may, for example, be substantially free of other isomers, or may be admixed, for example, as racemates or with all other, or other selected, stereoisomers. Chiral center(s) in a compound of the present invention can have the S or R configuration as defined by the IUPAC 1974 Recommendations. Further, to the extent a compound described herein may exist as a atropisomer (e.g., substituted biaryls), all forms of such atropisomer are considered part of this invention.

As used herein, the terms “subject” and “patient” are used interchangeable and refer to organisms to be treated by the methods of the present invention. Such organisms preferably include, but are not limited to, mammals (e.g., murines, simians, equines, bovines, porcines, canines, felines, and the like), and most preferably includes humans.

The term “EC₅₀” is art-recognized and refers to the concentration of a compound that is required to achieve 50% of the maximum possible activation of the target.

As used herein, the term “effective amount” refers to the amount of a compound sufficient to effect beneficial or desired results (e.g., a therapeutic, ameliorative, inhibitory or preventative result). An effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for diagnostic or therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see e.g., Martin, Remington's Pharmaceutical Sciences, 15th Ed., Mack Publ. Co., Easton, Pa. [1975].

As used herein, the term “pharmaceutically acceptable salt” refers to any pharmaceutically acceptable salt (e.g., acid or base) of a compound of the present invention which, upon administration to a subject, is capable of providing a compound of this invention or an active metabolite or residue thereof. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₄⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate (also known as toluenesulfonate), undecanoate, and the like. Other examples of salts include anions of the compounds of the present invention compounded with a suitable cation such as Na⁺, NH₄⁺, and NW₄⁺ (wherein W is a C₁₋₄ alkyl group), and the like. Further examples of salts include, but are not limited to: ascorbate, borate, nitrate, phosphate, salicylate, and sulfate. Further, acids which are generally considered suitable for the formation of pharmaceutically useful salts from basic pharmaceutical compounds are discussed, for example, by P. Stahl et al., Camille G. (eds.) Handbook of Pharmaceutical Salts. Properties, Selection and Use. (2002) Zurich: Wiley-VCH; S. Berge et al., Journal of Pharmaceutical Sciences (1977) 66(1) 1-19; P. Gould, International J. of Pharmaceutics (1986) 33 201-217; Anderson et al., The Practice of Medicinal Chemistry (1996), Academic Press, New York; and in The Orange Book (Food & Drug Administration, Washington, D.C. on their website). These disclosures are incorporated herein by reference.

Additional exemplary basic salts include, but are not limited to: ammonium salts, alkali metal salts such as sodium, lithium, and potassium salts, alkaline earth metal salts such as calcium and magnesium salts, salts with organic bases (for example, organic amines) such as dicyclohexylamines, t-butyl amines, and salts with amino acids such as arginine, lysine and the like. Basic nitrogen-containing groups may be quarternized with agents such as lower alkyl halides (e.g., methyl, ethyl, and butyl chlorides, bromides and iodides), dialkyl sulfates (e.g., dimethyl, diethyl, and dibutyl sulfates), long chain halides (e.g., decyl, lauryl, and stearyl chlorides, bromides and iodides), aralkyl halides (e.g., benzyl and phenethyl bromides), and others.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The present invention includes the compounds of the invention in all their isolated forms (such as any solvates, hydrates, stereoisomers, and tautomers thereof). Further, the invention includes compounds in which one or more of the atoms may be artificially enriched in a particular isotope having the same atomic number, but an atomic mass or mass number different from the atomic mass or mass number predominantly found in nature. The present invention is meant to include all suitable isotopic variations of the compounds of the invention. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds can be prepared without undue experimentation by conventional techniques known to those skilled in the art or by processes analogous to those described in the Examples herein using appropriate isotopically-enriched reagents and/or intermediates.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

The terms “a” and “an” as used herein mean “one or more” and include the plural unless the context is inappropriate.

As a general matter, compositions specifying a percentage are by weight unless otherwise specified.

### I. Therapeutic Applications

The invention provides methods of treating a cancer selected from the group consisting of (i) a squamous cell carcinoma located in a patient's head and/or neck and (ii) a urothelial carcinoma. Various aspects and embodiments are described below.

One aspect of the invention provides a method of treating a cancer selected from the group consisting of (i) a squamous cell carcinoma located in a patient's head and/or neck and (ii) a urothelial carcinoma. The method comprises administering to a patient in need thereof a therapeutically effective amount of a compound of Formula I, to thereby treat the cancer, wherein Formula I is represented by:

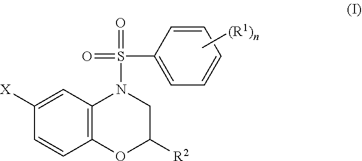

or a pharmaceutically acceptable salt thereof; wherein:
X is phenyl substituted by 1, 2, or 3 substituents independently selected from the group consisting of C₁₋₂ fluoroalkoxy, C₁₋₂ alkoxy, chloro, fluoro, and C₁₋₂ fluoroalkyl;
R¹ represents independently for each occurrence C₁₋₂ fluoroalkyl, chloro, fluoro, cyclopropyl, or C₁₋₃ alkyl;
R² is —(C₁₋₆ alkylene)—CO₂H; and
n is 1 or 2.

Another aspect of the invention provides a method of treating a cancer that is a squamous cell carcinoma located in a patient's head and/or neck, comprising administering to a patient in need thereof a therapeutically effective amount of a compound of Formula I, to thereby treat the cancer, wherein Formula I is represented by:

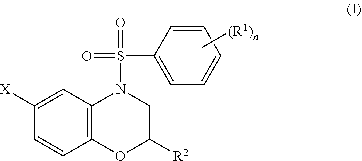

or a pharmaceutically acceptable salt thereof; wherein:
X is phenyl substituted by 1, 2, or 3 substituents independently selected from the group consisting of C₁₋₂ fluoroalkoxy, C₁₋₂ alkoxy, chloro, fluoro, and C₁₋₂ fluoroalkyl;
R¹ represents independently for each occurrence C₁₋₂ fluoroalkyl, chloro, fluoro, cyclopropyl, or C₁₋₃ alkyl;
R² is —(C₁₋₆ alkylene)—CO₂H; and
n is 1 or 2.

Another aspect of the invention provides a method of treating a cancer that is a urothelial carcinoma, comprising administering to a patient in need thereof a therapeutically effective amount of a compound of Formula I, to thereby treat the cancer, wherein Formula I is represented by:

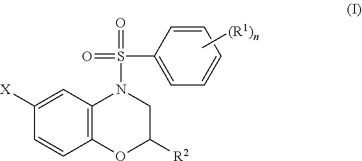

or a pharmaceutically acceptable salt thereof; wherein:
X is phenyl substituted by 1, 2, or 3 substituents independently selected from the group consisting of C₁₋₂ fluoroalkoxy, C₁₋₂ alkoxy, chloro, fluoro, and C₁₋₂ fluoroalkyl;
R¹ represents independently for each occurrence C₁₋₂ fluoroalkyl, chloro, fluoro, cyclopropyl, or C₁₋₃ alkyl;
R² is —(C₁₋₆ alkylene)—CO₂H; and
n is 1 or 2.

The methods may be further characterized according to additional features, such as the compound of Formula I, type of cancer, route of administration of the compound of Formula I, dose of compound of Formula I, dosing regimen for administering a compound of Formula I, and other features, as described in more detail below.

### Compound of Formula I

The definitions of variables in Formula I encompass multiple chemical groups. The application contemplates embodiments where, for example, i) the definition of a variable is a single chemical group selected from those chemical groups set forth above, ii) the definition of a variable is a collection of two or more of the chemical groups selected from those set forth above, and iii) the compound is defined by a combination of variables in which the variables are defined by (i) or (ii).

In certain embodiments, the compound is a pharmaceutically acceptable salt of Formula I. In certain other embodiments, the compound is a compound of Formula I.

The compound can be further characterized according to the definition of variables X, R¹, R², and n. Accordingly, in certain embodiments, X is phenyl substituted by C₁₋₂ fluoroalkoxy and fluoro. In certain embodiments, X is phenyl substituted by —OC(H)F₂ and fluoro.

In certain embodiments, R¹— is C₁₋₂ fluoroalkyl. In certain embodiments, R¹— is —CF₃.

In certain embodiments, R² is —(CH₂C(CH₃)₂)—CO₂H.

In certain embodiments, n is 1.

The description above describes multiple embodiments relating to compounds of Formula I. The patent application specifically contemplates all combinations of the embodiments.

In certain embodiments, the compound is

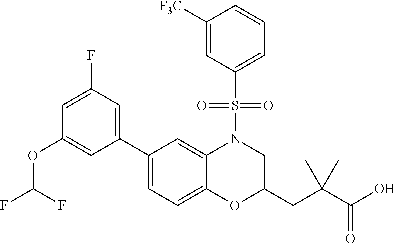 [SMILES: CC(C)(CC1CN(S(=O)(=O)c2cccc(C(F)(F)F)c2)c2cc(-c3cc(F)cc(OC(F)F)c3)ccc2O1)C(=O)O]

or a pharmaceutically acceptable salt thereof.

In certain embodiments, the compound is a pharmaceutically acceptable salt of

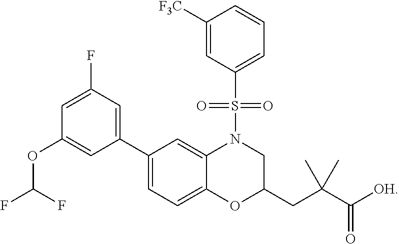 [SMILES: CC(C)(CC1CN(S(=O)(=O)c2cccc(C(F)(F)F)c2)c2cc(-c3cc(F)cc(OC(F)F)c3)ccc2O1)C(=O)O]

In certain embodiments, the compound is

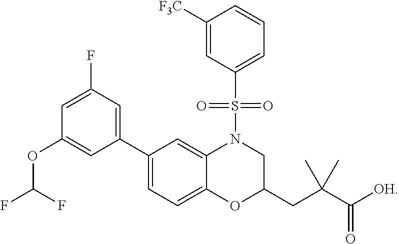 [SMILES: CC(C)(CC1CN(S(=O)(=O)c2cccc(C(F)(F)F)c2)c2cc(-c3cc(F)cc(OC(F)F)c3)ccc2O1)C(=O)O]

In certain embodiments, the compound is

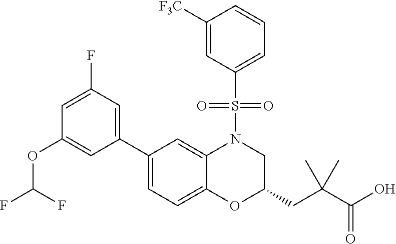 [SMILES: CC(C)(C[C@H]1CN(S(=O)(=O)c2cccc(C(F)(F)F)c2)c2cc(-c3cc(F)cc(OC(F)F)c3)ccc2O1)C(=O)O]

or a pharmaceutically acceptable salt thereof.

In certain embodiments, the compound is a pharmaceutically acceptable salt of

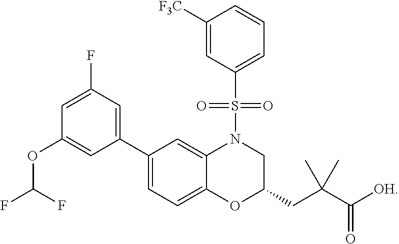 [SMILES: CC(C)(C[C@H]1CN(S(=O)(=O)c2cccc(C(F)(F)F)c2)c2cc(-c3cc(F)cc(OC(F)F)c3)ccc2O1)C(=O)O]

In certain embodiments, the compound is

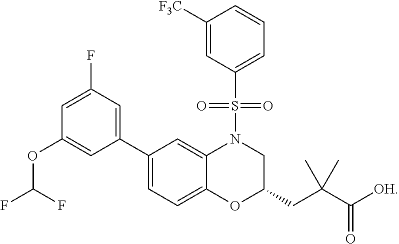 [SMILES: CC(C)(C[C@H]1CN(S(=O)(=O)c2cccc(C(F)(F)F)c2)c2cc(-c3cc(F)cc(OC(F)F)c3)ccc2O1)C(=O)O]

Compounds of Formula I may be prepared based on procedures described in Example 1 herein, which describes synthesis of sodium (S)-3-(6-(3-(difluoromethoxy)-5-fluorophenyl)-4-((3-(trifluoromethyl)phenyl)sulfonyl)-3,4-dihydro-2H-benzo[b][1,4]oxazin-2-yl)-2,2-dimethylpropanoate, which has the following formula:

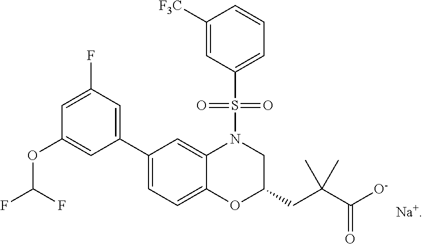 [SMILES: CC(C)(C[C@H]1CN(S(=O)(=O)c2cccc(C(F)(F)F)c2)c2cc(-c3cc(F)cc(OC(F)F)c3)ccc2O1)C(=O)[O-]]

(S)-3-(6-(3-(Difluoromethoxy)-5-fluorophenyl)-4-((3-(trifluoromethyl)phenyl)sulfonyl)-3,4-dihydro-2H-benzo[b][1,4]oxazin-2-yl)-2,2-dimethylpropanoic acid may be prepared by treating sodium (S)-3-(6-(3-(difluoromethoxy)-5-fluorophenyl)-4-((3-(trifluoromethyl)phenyl)sulfonyl)-3,4-dihydro-2H-benzo[b][1,4]oxazin-2-yl)-2,2-dimethylpropanoate with a mineral acid (e.g., HCl) to convert the carboxylate sodium salt to the free carboxylic acid.

### Type of Cancer

The method may be further characterized according to the type of cancer. Accordingly, in certain embodiments, the cancer is a squamous cell carcinoma located in a patient's head and/or neck. In certain embodiments, the squamous cell carcinoma is located in the patient's head. In certain embodiments, the squamous cell carcinoma is located in the patient's neck. In certain embodiments, the squamous cell carcinoma is located in the patient's head and neck. In certain embodiments, the squamous cell carcinoma is located in the patient's mouth or throat. In certain embodiments, the squamous cell carcinoma is located in the patient's throat. In certain embodiments, the squamous cell carcinoma is located in the patient's nasal cavity or nasopharynx.

In certain other embodiments, the cancer is a urothelial carcinoma. In certain embodiments, the urothelial carcinoma is located in the patient's bladder. In certain embodiments, the urothelial carcinoma is located in the patient's ureter or urethra. In certain embodiments, the urothelial carcinoma is located in patient's renal pelvis (i.e., the broadened top part of the ureter into which the kidney tubules drain).

### Route of Administration

The method may be further characterized according to the route of administration of the compound of Formula I. Accordingly, in certain embodiments, the compound of Formula I is administered orally to the patient.

### Dose

The method may be further characterized according to the dose of compound of Formula I administered to the patient. Accordingly, in certain embodiments, the compound is administered at a daily dose in the range of from about 100 mg/day to about 1200 mg/day. In certain embodiments, the compound is administered at a daily dose in the range of from about 100 mg/day to about 600 mg/day. In certain embodiments, the compound is administered at a daily dose in the range of from about 150 mg/day to about 500 mg/day. In certain embodiments, the compound is administered at a daily dose in the range of from about 200 mg/day to about 400 mg/day. In certain embodiments, the compound is administered at a daily dose in the range of from about 250 mg/day to about 350 mg/day. In certain embodiments, the compound is administered at a daily dose in the range of from about 275 mg/day to about 325 mg/day. In certain embodiments, the compound is administered at a daily dose in the range of from about 290 mg/day to about 310 mg/day. In certain embodiments, the compound is administered at a daily dose of about 300 mg/day.

In certain embodiments, the compound is administered at a daily dose in the range of from about 400 mg/day to about 500 mg/day. In certain embodiments, the compound is administered at a daily dose in the range of from about 425 mg/day to about 475 mg/day. In certain embodiments, the compound is administered at a daily dose in the range of from about 440 mg/day to about 460 mg/day. In certain embodiments, the compound is administered at a daily dose of about 450 mg/day.

In certain embodiments, the compound is administered at a daily dose in the range of from about 500 mg/day to about 600 mg/day, about 600 mg/day to about 700 mg/day, about 700 mg/day to about 800 mg/day, about 800 mg/day to about 900 mg/day, about 900 mg/day to about 1,000 mg/day, about 1,000 mg/day to about 1,100 mg/day, or about 1,100 mg/day to about 1,200 mg/day.

### Dosing Regimen

The method may be further characterized according to a dosing regimen for administering a compound of Formula I. Accordingly, in certain embodiments, the compound is administered at least once per day for at least 7 consecutive days. In certain embodiments, the compound is administered at least once per day for at least 14 consecutive days. In certain embodiments, the compound is administered at least once per day for at least 21 consecutive days. In certain embodiments, the compound is administered at least once per day for at least 28 consecutive days.

In certain embodiments, the compound is administered at least twice per day for at least 7 consecutive days. In certain embodiments, the compound is administered at least twice per day for at least 14 consecutive days. In certain embodiments, the compound is administered at least twice per day for at least 21 consecutive days. In certain embodiments, the compound is administered at least twice per day for at least 28 consecutive days.

In certain embodiments, the compound is administered twice per day for at least 7 consecutive days. In certain embodiments, the compound is administered twice per day for at least 14 consecutive days. In certain embodiments, the compound is administered twice per day for at least 21 consecutive days. In certain embodiments, the compound is administered twice per day for at least 28 consecutive days.

In certain embodiments, the compound is administered three times per day for at least 7 consecutive days. In certain embodiments, the compound is administered three times per day for at least 14 consecutive days. In certain embodiments, the compound is administered three times per day for at least 21 consecutive days. In certain embodiments, the compound is administered three times per day for at least 28 consecutive days.

In certain embodiments, the compound is administered on at least 10, 12, 14, 16, 18, 20, 22, 24, or 26 days in a 28-day cycle.

In yet other embodiments, a unit dosage of the compound is orally administered to the patient twice per day, the unit dosage contains from about 400 mg to about 500 mg of the compound, and wherein if the patient experiences an adverse event due to the compound, then thereafter for a period of at least 2 days the compound is orally administered to the patient at a reduced unit dose two times per day, wherein said reduced unit dose contains from about 300 mg to about 400 mg of the compound.

In yet other embodiments, a unit dosage of the compound is orally administered to the patient twice per day, the unit dosage contains from about 400 mg to about 500 mg of the compound, and wherein if the patient experiences an adverse event due to the compound, then thereafter for a period of at least 2 days the compound is orally administered to the patient at a reduced unit dose two times per day, wherein said reduced unit dose contains from about 300 mg to about 350 mg of the compound.

In yet other embodiments, a unit dosage of the compound is orally administered to the patient twice per day, the unit dosage contains from about 400 mg to about 500 mg of the compound, and wherein if the patient experiences an adverse event due to the compound, then thereafter for a period of at least 2 days the compound is orally administered to the patient at a reduced unit dose two times per day, wherein said reduced unit dose contains from about 340 mg to about 360 mg of the compound.

In yet other embodiments, a unit dosage of the compound is orally administered to the patient twice per day, the unit dosage contains from about 400 mg to about 500 mg of the compound, and wherein if the patient experiences an adverse event due to the compound, then thereafter for a period of at least 2 days the compound is orally administered to the patient at a reduced unit dose two times per day, wherein said reduced unit dose contains about 350 mg of the compound.

In yet other embodiments, a unit dosage of the compound is orally administered to the patient twice per day, the unit dosage contains from about 400 mg to about 500 mg of the compound, and wherein if the patient experiences an adverse event due to the compound, then thereafter for a period of at least 2 days the compound is orally administered to the patient at a reduced unit dose two times per day, wherein said reduced unit dose contains from about 300 mg to about 320 mg of the compound.

In yet other embodiments, a unit dosage of the compound is orally administered to the patient twice per day, the unit dosage contains from about 400 mg to about 500 mg of the compound, and wherein if the patient experiences an adverse event due to the compound, then thereafter for a period of at least 2 days the compound is orally administered to the patient at a reduced unit dose two times per day, wherein said reduced unit dose contains about 300 mg of the compound.

In certain embodiments, the unit dosage of the compound is orally administered to the patient two times per day for a duration of at least 1 weeks. In certain embodiments, the unit dosage of the compound is orally administered to the patient two times per day for a duration of at least 2 weeks. In certain embodiments, the unit dosage of the compound is orally administered to the patient two times per day for a duration of at least 4 weeks.

In certain embodiments, the reduced unit dose of the compound is orally administered to the patient two times per day for a duration of at least 7 days. In certain embodiments, the reduced unit dose of the compound is orally administered to the patient two times per day for a duration of at least 2 weeks. In certain embodiments, the reduced unit dose of the compound is orally administered to the patient two times per day for a duration of at least 3 weeks. In certain embodiments, the reduced unit dose of the compound is orally administered to the patient two times per day for a duration of at least 4 weeks. In certain embodiments, the reduced unit dose of the compound is orally administered to the patient two times per day for a duration of at least 6 weeks. In certain embodiments, the reduced unit dose of the compound is orally administered to the patient two times per day for a duration of at least 8 weeks.

In certain embodiments, the adverse event is gastric distress. In certain embodiments, the adverse event is diarrhea. In certain embodiments, the adverse event is one or more of fatigue, anemia, poor appetite, or dry mouth.

Reduction in Size and/or Number of Occurrences of the Cancer

The method may be further characterized according to the reduction in size and/or reduction in the number of occurrences of the cancer due to administering the compound of Formula I. Accordingly, in certain embodiments, there is at least a 20% reduction in the size of said cancer. In certain embodiments, there is at least a 35% reduction in the size of said cancer. In certain embodiments, there is at least a 50% reduction in the size of said cancer.

In certain embodiments, there is at least a 20% reduction in the number of occurrences of said cancer. In certain embodiments, there is at least a 35% reduction in the number of occurrences of said cancer. In certain embodiments, there is at least a 50% reduction in the number of occurrences of said cancer.

The reduction in size of said cancer and/or reduction in number of occurrences of said cancer may be measured at, for example, 1, 2, 3, 4, 5, 6, 7, 8, 9, 10, 11, or 12 weeks, or later after initiation of therapy (i.e., first administration of a compound of Formula as part of a dosing regimen designed to treat said cancer).

### Identity of the Patient

The method may be further characterized according to the identity of the patient. Accordingly, in certain embodiments, the patient is an adult human. In certain other embodiments, the patient is a pediatric human.

### Form of Administration

The method may be further characterized according to the form in which the said compound of Formula I is administered to the patient. Accordingly, in certain embodiments, the compound is administered in the form of a pharmaceutical composition comprising a pharmaceutically acceptable carrier and the compound.

### Stereochemical Purity of the Compound of Formula I

The method may be further characterized according to the stereochemical purity of the compound of Formula I administered to the patient. For example, in certain embodiments, the compound has an enantiomeric excess of at least 80%, 85%, 90%, 95%, 97%, 98%, or 99%. In certain embodiments, the compound has an enantiomeric excess of at least 95%. In certain embodiments, the compound has an enantiomeric excess of at least 98%.

### EXEMPLARY EMBODIMENTS

The invention is further illustrated by the following exemplary embodiments:

### Embodiment 1

A method of treating a cancer selected from the group consisting of (i) a squamous cell carcinoma located in a patient's head and/or neck and (ii) a urothelial carcinoma, comprising administering to a patient in need thereof a therapeutically effective amount of a compound of Formula I, to thereby treat the cancer, wherein Formula I is represented by:

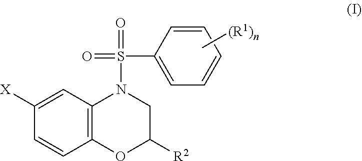

or a pharmaceutically acceptable salt thereof; wherein:
X is phenyl substituted by 1, 2, or 3 substituents independently selected from the group consisting of C₁₋₂ fluoroalkoxy, C₁₋₂ alkoxy, chloro, fluoro, and C₁₋₂ fluoroalkyl;
R¹— represents independently for each occurrence C₁₋₂ fluoroalkyl, chloro, fluoro, cyclopropyl, or C₁₋₃ alkyl;
R² is —(C₁₋₆ alkylene)—CO₂H; and
n is 1 or 2.

### Embodiment 2

The method of embodiment 1, wherein X is phenyl substituted by C₁₋₂ fluoroalkoxy and fluoro.

### Embodiment 3

The method of embodiment 1, wherein X is phenyl substituted by —OC(H)F₂ and fluoro.

### Embodiment 4

The method of any one of embodiments 1-3, wherein R¹ is C₁₋₂ fluoroalkyl.

### Embodiment 5

The method of any one of embodiments 1-3, wherein R¹ is —CF₃.

### Embodiment 6

The method of any one of embodiments 1-5, wherein R² is —(C₋H₂C(CH₃)₂)—CO₂H.

### Embodiment 7

The method of any one of embodiments 1-6, wherein n is 1.

### Embodiment 8

The method of embodiment 1, wherein the compound is

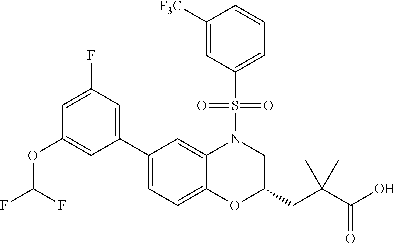 [SMILES: CC(C)(C[C@H]1CN(S(=O)(=O)c2cccc(C(F)(F)F)c2)c2cc(-c3cc(F)cc(OC(F)F)c3)ccc2O1)C(=O)O]

or a pharmaceutically acceptable salt thereof.

### Embodiment 9

The method of embodiment 1, wherein the compound is a pharmaceutically acceptable salt of

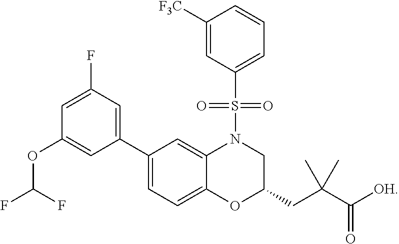 [SMILES: CC(C)(C[C@H]1CN(S(=O)(=O)c2cccc(C(F)(F)F)c2)c2cc(-c3cc(F)cc(OC(F)F)c3)ccc2O1)C(=O)O]

### Embodiment 10

The method of embodiment 1, wherein the compound is

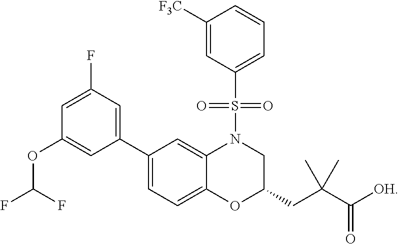 [SMILES: CC(C)(C[C@H]1CN(S(=O)(=O)c2cccc(C(F)(F)F)c2)c2cc(-c3cc(F)cc(OC(F)F)c3)ccc2O1)C(=O)O]

### Embodiment 11

The method of any one of embodiments 1-10, wherein the cancer is a squamous cell carcinoma located in a patient's head and/or neck.

### Embodiment 12

The method of embodiment 11, wherein the squamous cell carcinoma is located in the patient's head.

### Embodiment 13

The method of embodiment 11, wherein the squamous cell carcinoma is located in the patient's neck.

### Embodiment 14

The method of embodiment 11, wherein the squamous cell carcinoma is located in the patient's head and neck.

### Embodiment 15

The method of embodiment 11, wherein the squamous cell carcinoma is located in the patient's mouth or throat.

### Embodiment 16

The method of embodiment 11, wherein the squamous cell carcinoma is located in the patient's nasal cavity or nasopharynx.

### Embodiment 17

The method of any one of embodiments 1-10, wherein the cancer is a urothelial carcinoma.

### Embodiment 18

The method of embodiment 17, wherein the urothelial carcinoma is located in the patient's bladder.

### Embodiment 19

The method of embodiment 17, wherein the urothelial carcinoma is located in the patient's ureter or urethra.

### Embodiment 20

The method of any one of embodiments 1-19, wherein the compound is administered orally to the patient.

### Embodiment 21

The method of any one of embodiments 1-20, wherein the compound is administered at a daily dose in the range of from about 100 mg/day to about 600 mg/day.

### Embodiment 22

The method of any one of embodiments 1-20, wherein the compound is administered at a daily dose in the range of from about 150 mg/day to about 500 mg/day.

### Embodiment 23

The method of any one of embodiments 1-20, wherein the compound is administered at a daily dose in the range of from about 200 mg/day to about 400 mg/day.

### Embodiment 24

The method of any one of embodiments 1-20, wherein the compound is administered at a daily dose in the range of from about 250 mg/day to about 350 mg/day.

### Embodiment 25

The method of any one of embodiments 1-20, wherein the compound is administered at a daily dose in the range of from about 275 mg/day to about 325 mg/day.

### Embodiment 26

The method of any one of embodiments 1-20, wherein the compound is administered at a daily dose in the range of from about 290 mg/day to about 310 mg/day.

### Embodiment 27

The method of any one of embodiments 1-20, wherein the compound is administered at a daily dose of about 300 mg/day.

### Embodiment 28

The method of any one of embodiments 1-20, wherein the compound is administered at a daily dose in the range of from about 400 mg/day to about 500 mg/day.

### Embodiment 29

The method of any one of embodiments 1-20, wherein the compound is administered at a daily dose in the range of from about 425 mg/day to about 475 mg/day.

### Embodiment 30

The method of any one of embodiments 1-20, wherein the compound is administered at a daily dose in the range of from about 440 mg/day to about 460 mg/day.

### Embodiment 31

The method of any one of embodiments 1-20, wherein the compound is administered at a daily dose of about 450 mg/day.

### Embodiment 32

The method of any one of embodiments 1-31, wherein the compound is administered at least once per day for at least 7 consecutive days.

### Embodiment 33

The method of any one of embodiments 1-31, wherein the compound is administered at least once per day for at least 14 consecutive days.

### Embodiment 34

The method of any one of embodiments 1-31, wherein the compound is administered at least once per day for at least 21 consecutive days.

### Embodiment 35

The method of any one of embodiments 1-31, wherein the compound is administered at least once per day for at least 28 consecutive days.

### Embodiment 36

The method of any one of embodiments 1-31, wherein the compound is administered at least twice per day for at least 7 consecutive days.

### Embodiment 37

The method of any one of embodiments 1-31, wherein the compound is administered at least twice per day for at least 14 consecutive days.

### Embodiment 38

The method of any one of embodiments 1-31, wherein the compound is administered at least twice per day for at least 21 consecutive days.

### Embodiment 39

The method of any one of embodiments 1-31, wherein the compound is administered at least twice per day for at least 28 consecutive days.

### Embodiment 40

The method of any one of embodiments 1-31, wherein the compound is administered twice per day for at least 7 consecutive days.

### Embodiment 41

The method of any one of embodiments 1-31, wherein the compound is administered twice per day for at least 14 consecutive days.

### Embodiment 42

The method of any one of embodiments 1-31, wherein the compound is administered twice per day for at least 21 consecutive days.

### Embodiment 43

The method of any one of embodiments 1-31, wherein the compound is administered twice per day for at least 28 consecutive days.

### Embodiment 44

The method of any one of embodiments 1-19, wherein a unit dosage of the compound is orally administered to the patient twice per day, the unit dosage contains from about 400 mg to about 500 mg of the compound, and wherein if the patient experiences an adverse event due to the compound, then thereafter for a period of at least 2 days the compound is orally administered to the patient at a reduced unit dose two times per day, wherein said reduced unit dose contains from about 300 mg to about 400 mg of the compound.

### Embodiment 45

The method of any one of embodiments 1-19, wherein a unit dosage of the compound is orally administered to the patient twice per day, the unit dosage contains from about 400 mg to about 500 mg of the compound, and wherein if the patient experiences an adverse event due to the compound, then thereafter for a period of at least 2 days the compound is orally administered to the patient at a reduced unit dose two times per day, wherein said reduced unit dose contains from about 300 mg to about 350 mg of the compound.

### Embodiment 46

The method of any one of embodiments 1-19, wherein a unit dosage of the compound is orally administered to the patient twice per day, the unit dosage contains from about 400 mg to about 500 mg of the compound, and wherein if the patient experiences an adverse event due to the compound, then thereafter for a period of at least 2 days the compound is orally administered to the patient at a reduced unit dose two times per day, wherein said reduced unit dose contains from about 340 mg to about 360 mg of the compound.

### Embodiment 47

The method of any one of embodiments 1-19, wherein a unit dosage of the compound is orally administered to the patient twice per day, the unit dosage contains from about 400 mg to about 500 mg of the compound, and wherein if the patient experiences an adverse event due to the compound, then thereafter for a period of at least 2 days the compound is orally administered to the patient at a reduced unit dose two times per day, wherein said reduced unit dose contains about 350 mg of the compound.

### Embodiment 48

The method of any one of embodiments 1-19, wherein a unit dosage of the compound is orally administered to the patient twice per day, the unit dosage contains from about 400 mg to about 500 mg of the compound, and wherein if the patient experiences an adverse event due to the compound, then thereafter for a period of at least 2 days the compound is orally administered to the patient at a reduced unit dose two times per day, wherein said reduced unit dose contains from about 300 mg to about 320 mg of the compound.

### Embodiment 49

The method of any one of embodiments 1-19, wherein a unit dosage of the compound is orally administered to the patient twice per day, the unit dosage contains from about 400 mg to about 500 mg of the compound, and wherein if the patient experiences an adverse event due to the compound, then thereafter for a period of at least 2 days the compound is orally administered to the patient at a reduced unit dose two times per day, wherein said reduced unit dose contains about 300 mg of the compound.

### Embodiment 50

The method of any one of embodiments 44-49, wherein the unit dosage of the compound is orally administered to the patient two times per day for a duration of at least 1 weeks.

### Embodiment 51

The method of any one of embodiments 44-49, wherein the unit dosage of the compound is orally administered to the patient two times per day for a duration of at least 2 weeks.

### Embodiment 52

The method of any one of embodiments 44-49, wherein the unit dosage of the compound is orally administered to the patient two times per day for a duration of at least 4 weeks.

### Embodiment 53

The method of any one of embodiments 44-52, wherein the reduced unit dose of the compound is orally administered to the patient two times per day for a duration of at least 7 days.

### Embodiment 54

The method of any one of embodiments 44-52, wherein the reduced unit dose of the compound is orally administered to the patient two times per day for a duration of at least 2 weeks.

### Embodiment 55

The method of any one of embodiments 44-52, wherein the reduced unit dose of the compound is orally administered to the patient two times per day for a duration of at least 3 weeks.

### Embodiment 56

The method of any one of embodiments 44-55, wherein the adverse event is gastric distress.

### Embodiment 57

The method of any one of embodiments 44-55, wherein the adverse event is diarrhea.

### Embodiment 58

The method of any one of embodiments 44-55, wherein the adverse event is one or more of fatigue, anemia, poor appetite, or dry mouth.

### Embodiment 59

The method of any one of embodiments 1-58, wherein there is at least a 20% reduction in the size of said cancer.

### Embodiment 60

The method of any one of embodiments 1-58, wherein there is at least a 35% reduction in the size of said cancer.

### Embodiment 61

The method of any one of embodiments 1-58, wherein there is at least a 50% reduction in the size of said cancer.

### Embodiment 62

The method of any one of embodiments 1-61, wherein there is at least a 20% reduction in the number of occurrences of said cancer.

### Embodiment 63

The method of any one of embodiments 1-61, wherein there is at least a 35% reduction in the number of occurrences of said cancer.

### Embodiment 64

The method of any one of embodiments 1-61, wherein there is at least a 50% reduction in the number of occurrences of said cancer.

### Embodiment 65

The method of any one of embodiments 1-64, wherein the patient is an adult human.

### Embodiment 66

The method of any one of embodiments 1-64, wherein the patient is a pediatric human.

### Embodiment 67

The method of any one of embodiments 1-66, wherein the compound is administered in the form of a pharmaceutical composition comprising a pharmaceutically acceptable carrier and the compound.

### II. Combination Therapy

Another aspect of the invention provides for combination therapy. Compounds of Formula I and their pharmaceutically acceptable salts may be used in combination with additional therapeutic agents to treat medical disorders described herein.

Exemplary therapeutic agents that may be used as part of a combination therapy in treating cancer, include, for example, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, and leutinizing hormone releasing factor.

An additional class of agents that may be used as part of a combination therapy in treating cancer is immune checkpoint inhibitors (also referred to as immune checkpoint blockers). Immune checkpoint inhibitors are a class of therapeutic agents that have the effect of blocking immune checkpoints, and thereby result in activating the body's anti-tumor immune system. See, for example, Pardoll in Nature Reviews Cancer (2012) vol. 12, pages 252-264. Exemplary immune checkpoint inhibitors include agents that inhibit one or more of (i) cytotoxic T-lymphocyte-associated antigen 4 (CTLA4), (ii) programmed cell death protein 1 (PD1), (iii) PDL1, (iv) LAG-3, (v) B7-H3, (vi) B7-H4, and (vii) TIM3. The CTLA4 inhibitor Ipilumumab has been approved by the United States Food and Drug Administration for treating melanoma.

Yet other agents that may be used as part of a combination therapy in treating cancer are monoclonal antibody agents that target non-checkpoint targets (e.g., herceptin) and non-cytotoxic agents (e.g., tyrosine-kinase inhibitors such as bevacizumab and ramucirumab).

Accordingly, another aspect of the invention provides a method of treating a cancer selected from the group consisting of (i) a squamous cell carcinoma located in a patient's head and/or neck and (ii) a urothelial carcinoma, where the method comprises administering to the patient in need thereof (i) a therapeutically effective amount of a RORγ agonist compound described herein (e.g., a compound of Formula I) and (ii) a second anti-cancer agent, in order to treat the cancer, where the second therapeutic agent may be one of the additional therapeutic agents described above (e.g., mitomycin, tretinoin, ribomustin, gemcitabine, an immune checkpoint inhibitor, or a monoclonal antibody agent that targets non-checkpoint targets) or one of the following:
an inhibitor selected from an ALK Inhibitor, an ATR Inhibitor, an A2A Antagonist, a Base Excision Repair Inhibitor, a Bcr-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor;
an agonist of OX40, CD137, CD40, GITR, CD27, HVEM, TNFRSF25, or ICOS;
a therapeutic antibody or other protein targeting one of the following: CD20, CD30, CD33, CD52, EpCAM, CEA, gpA33, a mucin, TAG-72, CAIX, PSMA, a folate-binding protein, a ganglioside, Le, VEGF, VEGFR, VEGFR2, integrin αVβ3, integrin α5β1, EGFR, ERBB2, ERBB3, MET, IGF1R, EPHA3, TRAILR1, TRAILR2, RANKL, FAP, tenascin, CD19, KIR, NKG2A, CD47, CEACAM1, c-MET, VISTA, CD73, CD38, BAFF, B4GALNT1, and IL-17;
a cytokine selected from IL-12, IL-15, GM-CSF, G-CSF, IL-2, and Type 1 IFN family;
a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-1-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine; or
a placental growth factor, an antibody-drug conjugate, an oncolytic virus, or an anti-cancer vaccine.

In certain embodiments, the second anti-cancer agent is an ALK Inhibitor. In certain embodiments, the second anti-cancer agent is an ALK Inhibitor comprisng ceritinib or crizotinib. In certain embodiments, the second anti-cancer agent is an ATR Inhibitor. In certain embodiments, the second anti-cancer agent is an ATR Inhibitor comprising AZD6738 or VX-970. In certain embodiments, the second anti-cancer agent is an A2A Antagonist. In certain embodiments, the second anti-cancer agent is a Base Excision Repair Inhibitor comprising methoxyamine. In certain embodiments, the second anti-cancer agent is a Base Excision Repair Inhibitor, such as methoxyamine. In certain embodiments, the second anti-cancer agent is a Bcr-Abl Tyrosine Kinase Inhibitor. In certain embodiments, the second anti-cancer agent is a Bcr-Abl Tyrosine Kinase Inhibitor comprising dasatinib or nilotinib. In certain embodiments, the second anti-cancer agent is a Bruton's Tyrosine Kinase Inhibitor. In certain embodiments, the second anti-cancer agent is a Bruton's Tyrosine Kinase Inhibitor comprising ibrutinib. In certain embodiments, the second anti-cancer agent is a CDC7 Inhibitor. In certain embodiments, the second anti-cancer agent is a CDC7 Inhibitor comprising RXDX-103 or AS-141.

In certain embodiments, the second anti-cancer agent is a CHK1 Inhibitor. In certain embodiments, the second anti-cancer agent is a CHK1 Inhibitor comprising MK-8776, ARRY-575, or SAR-020106. In certain embodiments, the second anti-cancer agent is a Cyclin-Dependent Kinase Inhibitor. In certain embodiments, the second anti-cancer agent is a Cyclin-Dependent Kinase Inhibitor comprising palbociclib. In certain embodiments, the second anti-cancer agent is a DNA-PK Inhibitor. In certain embodiments, the second anti-cancer agent is a DNA-PK Inhibitor comprising MSC2490484A. In certain embodiments, the second anti-cancer agent is Inhibitor of both DNA-PK and mTOR. In certain embodiments, the second anti-cancer agent comprises CC-115.

In certain embodiments, the second anti-cancer agent is a DNMT1 Inhibitor. In certain embodiments, the second anti-cancer agent is a DNMT1 Inhibitor comprising decitabine, RX-3117, guadecitabine, NUC-8000, or azacytidine. In certain embodiments, the second anti-cancer agent comprises a DNMT1 Inhibitor and 2-chloro-deoxyadenosine. In certain embodiments, the second anti-cancer agent comprises ASTX-727.

In certain embodiments, the second anti-cancer agent is a HDAC Inhibitor. In certain embodiments, the second anti-cancer agent is a HDAC Inhibitor comprising OBP-801, CHR-3996, etinostate, resminostate, pracinostat, CG-200745, panobinostat, romidepsin, mocetinostat, belinostat, AR-42, ricolinostat, KA-3000, or ACY-241.

In certain embodiments, the second anti-cancer agent is a Hedgehog Signaling Pathway Inhibitor. In certain embodiments, the second anti-cancer agent is a Hedgehog Signaling Pathway Inhibitor comprising sonidegib or vismodegib. In certain embodiments, the second anti-cancer agent is an IDO Inhibitor. In certain embodiments, the second anti-cancer agent is an IDO Inhibitor comprising INCB024360. In certain embodiments, the second anti-cancer agent is a JAK Inhibitor. In certain embodiments, the second anti-cancer agent is a JAK Inhibitor comprising ruxolitinib or tofacitinib. In certain embodiments, the second anti-cancer agent is a mTOR Inhibitor. In certain embodiments, the second anti-cancer agent is a mTOR Inhibitor comprising everolimus or temsirolimus. In certain embodiments, the second anti-cancer agent is a MEK Inhibitor. In certain embodiments, the second anti-cancer agent is a MEK Inhibitor comprising cobimetinib or trametinib. In certain embodiments, the second anti-cancer agent is a MELK Inhibitor. In certain embodiments, the second anti-cancer agent is a MELK Inhibitor comprising ARN-7016, APTO-500, or OTS-167. In certain embodiments, the second anti-cancer agent is a MTH1 Inhibitor. In certain embodiments, the second anti-cancer agent is a MTH1 Inhibitor comprising (S)-crizotinib, TH287, or TH588.

In certain embodiments, the second anti-cancer agent is a PARP Inhibitor. In certain embodiments, the second anti-cancer agent is a PARP Inhibitor comprising MP-124, olaparib, BGB-290, talazoparib, veliparib, niraparib, E7449, rucaparb, or ABT-767. In certain embodiments, the second anti-cancer agent is a Phosphoinositide 3-Kinase Inhibitor. In certain embodiments, the second anti-cancer agent is a Phosphoinositide 3-Kinase Inhibitor comprising idelalisib. In certain embodiments, the second anti-cancer agent is an inhibitor of both PARP1 and DHODH (i.e., an agent that inhibits both poly ADP ribose polymerase 1 and dihydroorotate dehydrogenase).

In certain embodiments, the second anti-cancer agent is a Proteasome Inhibitor. In certain embodiments, the second anti-cancer agent is a Proteasome Inhibitor comprising bortezomib or carfilzomib. In certain embodiments, the second anti-cancer agent is a Topoisomerase-II Inhibitor. In certain embodiments, the second anti-cancer agent is a Topoisomerase-II Inhibitor comprising vosaroxin.

In certain embodiments, the second anti-cancer agent is a Tyrosine Kinase Inhibitor. In certain embodiments, the second anti-cancer agent is a Tyrosine Kinase Inhibitor comprising bosutinib, cabozantinib, imatinib or ponatinib. In certain embodiments, the second anti-cancer agent is a VEGFR Inhibitor. In certain embodiments, the second anti-cancer agent is a VEGFR Inhibitor comprising regorafenib. In certain embodiments, the second anti-cancer agent is a WEE1 Inhibitor. In certain embodiments, the second anti-cancer agent is a WEE1 Inhibitor comprising AZD1775.

In certain embodiments, the second anti-cancer agent is an agonist of OX40, CD137, CD40, GITR, CD27, HVEM, TNFRSF25, or ICOS. In certain embodiments, the second anti-cancer agent is a therapeutic antibody selected from the group consisting of rituximab, ibritumomab tiuxetan, tositumomab, obinutuzumab, ofatumumab, brentuximab vedotin, gemtuzumab ozogamicin, alemtuzumab, IGN101, adecatumumab, labetuzumab, huA33, pemtumomab, oregovomab, minetumomab, cG250, J591, Mov18, farletuzumab, 3F8, ch14.18, KW-2871, hu3S193, lgN311, bevacizumab, IM-2C6, pazopanib, sorafenib, axitinib, CDP791, lenvatinib, ramucirumab, etaracizumab, volociximab, cetuximab, panitumumab, nimotuzumab, 806, afatinib, erlotinib, gefitinib, osimertinib, vandetanib, trastuzumab, pertuzumab, MM-121, AMG 102, METMAB, SCH 900105, AVE1642, IMC-A12, MK-0646, R1507, CP 751871, KB004, IIIA-4, mapatumumab, HGS-ETR2, CS-1008, denosumab, sibrotuzumab, F19, 8106, MEDI551, lirilumab, MEDI9447, daratumumab, belimumab, canakinumab, dinutuximab, siltuximab, and tocilizumab.

In certain embodiments, the second anti-cancer agent is a placental growth factor. In certain embodiments, the second anti-cancer agent is a placental growth factor comprising ziv-aflibercept. In certain embodiments, the second anti-cancer agent is an antibody-drug conjugate. In certain embodiments, the second anti-cancer agent is an antibody-drug conjugate selected from the group consisting of brentoxumab vedotin and trastuzumab emtransine.

In certain embodiments, the second anti-cancer agent is an oncolytic virus. In certain embodiments, the second anti-cancer agent is the oncolytic virus talimogene laherparepvec. In certain embodiments, the second anti-cancer agent is an anti-cancer vaccine. In certain embodiments, the second anti-cancer agent is an anti-cancer vaccine directed to head and neck squamous cell carcinoma. In certain embodiments, the second anti-cancer agent is an anti-cancer vaccine directed to urothelial cancer. In certain embodiments, the second anti-cancer agent is an anti-cancer vaccine selected from the group consisting of a GM-CSF tumor vaccine, a STING/GM-CSF tumor vaccine, and NY-ESO-1. In certain embodiments, the second anti-cancer agent is a cytokine selected from IL-12, IL-15, GM-CSF, and G-CSF.

In certain embodiments, the second anti-cancer agent is a therapeutic agent selected from sipuleucel-T, aldesleukin (a human recombinant interleukin-2 product having the chemical name des-alanyl-1, serine-125 human interleukin-2), dabrafenib (a kinase inhibitor having the chemical name N-{3-[5-(2-aminopyrimidin-4-yl)-2-tert-butyl-1,3-thiazol-4-yl]-2-fluorophenyl}-2,6-difluorobenzenesulfonamide), vemurafenib (a kinase inhibitor having the chemical name propane-1-sulfonic acid {3-[5-(4-chlorophenyl)-1H-pyrrolo[2,3-b]pyridine-3-carbonyl]-2,4-difluoro-phenyl}-amide), and 2-chloro-deoxyadenosine.

In certain embodiments, the second anti-cancer agent is radiation therapy.

In certain embodiments, the method further comprises administering a third anti-cancer agent, which may be, for example, radiation therapy.

The amount of compound of Formula I (or pharmaceutically acceptable salt thereof) and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like

In certain embodiments, the compound of Formula I (or pharmaceutically acceptable salt thereof) and the additional therapeutic agent(s) may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

### III. Pharmaceutical Compositions

As indicated above, the invention provides pharmaceutical compositions, which comprise one or more of the compounds described above, formulated together with one or more pharmaceutically acceptable carriers (additives) and/or diluents. The pharmaceutical compositions may be specially formulated for administration in solid or liquid form, including those adapted for the following: (1) oral administration, for example, drenches (aqueous or non-aqueous solutions or suspensions), tablets, e.g., those targeted for buccal, sublingual, and systemic absorption, boluses, powders, granules, pastes for application to the tongue; (2) parenteral administration, for example, by subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Accordingly, one aspect of the invention provides a pharmaceutical composition comprising a compound of Formula I and a pharmaceutically acceptable carrier. In certain embodiments, the invention provides a pharmaceutical composition comprising a pharmaceutically acceptable carrier and

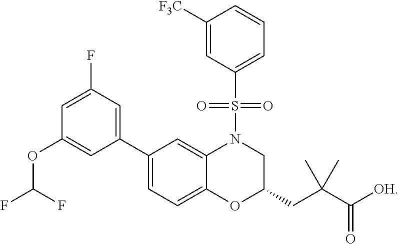 [SMILES: CC(C)(C[C@H]1CN(S(=O)(=O)c2cccc(C(F)(F)F)c2)c2cc(-c3cc(F)cc(OC(F)F)c3)ccc2O1)C(=O)O]

In certain embodiments, the invention provides a pharmaceutical composition comprising (i) a pharmaceutically acceptable carrier and (ii)

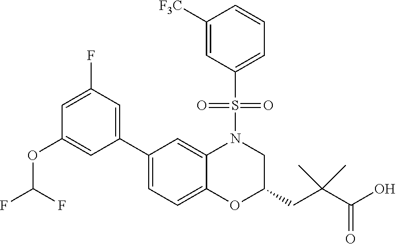 [SMILES: CC(C)(C[C@H]1CN(S(=O)(=O)c2cccc(C(F)(F)F)c2)c2cc(-c3cc(F)cc(OC(F)F)c3)ccc2O1)C(=O)O]

or a pharmaceutically acceptable salt thereof.

In certain embodiments, the pharmaceutical composition comprising a therapeutically effective amount of said compound and a pharmaceutically acceptable carrier. The phrase “therapeutically effective amount” as used herein means that amount of a compound, material, or composition comprising a compound of the present invention which is effective for producing some desired therapeutic effect in at least a sub-population of cells in an animal at a reasonable benefit/risk ratio applicable to any medical treatment.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders orally bioavailable a compound of the present invention.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to humans and animals, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Oral administrations are preferred.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrases “systemic administration,” “administered systemically,” “peripheral administration” and “administered peripherally” as used herein mean the administration of a compound, drug or other material other than directly into the central nervous system, such that it enters the patient's system and, thus, is subject to metabolism and other like processes, for example, subcutaneous administration.

These compounds may be administered to humans and other animals for therapy by any suitable route of administration, including orally, nasally, as by, for example, a spray, rectally, intravaginally, parenterally, intracisternally and topically, as by powders, ointments or drops, including buccally and sublingually.

Regardless of the route of administration selected, the compounds of the present invention, which may be used in a suitable hydrated form, and/or the pharmaceutical compositions of the present invention, are formulated into pharmaceutically-acceptable dosage forms by conventional methods known to those of skill in the art.

Actual dosage levels of the active ingredients in the pharmaceutical compositions of this invention may be varied so as to obtain an amount of the active ingredient which is effective to achieve the desired therapeutic response for a particular patient, composition, and mode of administration, without being toxic to the patient.

The selected dosage level will depend upon a variety of factors including the activity of the particular compound of the present invention employed, or the ester, salt or amide thereof, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

If desired, the effective daily dose of the active compound may be administered as two, three, four, five, six or more sub-doses administered separately at appropriate intervals throughout the day, optionally, in unit dosage forms. Preferred dosing is one administration per day.

The invention further provides a unit dosage form (such as a tablet or capsule) comprising an aryl dihydro-2H-benzo[b][1,4]oxazine sulfonamide compound described herein in a therapeutically effective amount for the treatment of a medical disorder described herein.

The description above describes multiple aspects and embodiments of the invention. The patent application specifically contemplates all combinations and permutations of the aspects and embodiments.

### IV. Medical Kits

Another aspect of this invention is a kit comprising (i) a compound of Formula I or a pharmaceutically acceptable salt thereof, and (ii) instructions for treating a cancer selected from the group consisting of (i) a squamous cell carcinoma located in a patient's head and/or neck and (ii) a urothelial carcinoma.

### EXAMPLES

The invention now being generally described, will be more readily understood by reference to the following examples, which are included merely for purposes of illustration of certain aspects and embodiments of the present invention, and is not intended to limit the invention.

### Example 1—Synthesis of sodium (S)-3-(6-(3-(difluoromethoxy)-5-fluorophenyl)-4-((3-(trifluoromethyl)phenyl)sulfonyl)-3,4-dihydro-2H-benzo[b][1,4]oxazin-2-yl)-2,2-dimethylpropanoate

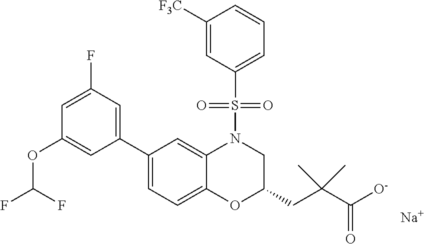 [SMILES: CC(C)(C[C@H]1CN(S(=O)(=O)c2cccc(C(F)(F)F)c2)c2cc(-c3cc(F)cc(OC(F)F)c3)ccc2O1)C(=O)[O-] ; [Na+]]

### Part I—Synthesis of (S)-2,2-dimethyl-3-(oxiran-2-yl)propanenitrile

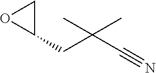

A 2.5M solution of n-butyl lithium (40 mL, 100 mmol) was added dropwise to a stirred solution of diisopropylamine (14.1 mL, 100 mmol) in THF (200 mL) at 0° C. The solution was stirred at 0° C. for thirty minutes. The cold solution was added dropwise to a solution of (S)-2-(chloromethyl)oxirane (9.3 g, 100 mmol), 2-methylpropanenitrile (6.9 g, 100 mmol), and tetrahydrofuran (200 mL) at −45° C. The mixture was stirred at −45° C., and then allowed to warm up to room temperature overnight. The mixture was quenched by the addition of saturated ammonium chloride (100 mL), and extracted with ethyl acetate (100 mL). The organic layers were washed with water (100 mL), dried (Na₂SO₄), filtered and concentrated to afford (S)-2,2-dimethyl-3-(oxiran-2-yl)propanenitrile (10.1 g, 81%) as a light yellow oil.

### Part II—Synthesis of (S)-5-(chloromethyl)-3,3-dimethyl-tetrahydrofuran-2-one

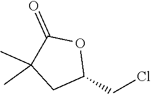

(S)-2,2-dimethyl-3-(oxiran-2-yl)propanenitrile (10.1 g, 80.7 mmol) was dissolved in concentrated hydrogen chloride (20 mL) and stirred at room temperature for ten minutes. The solution was diluted with acetic acid (40 mL) and then heated to 50° C. overnight. The reaction mixture was partitioned between water (50 mL) and ethyl acetate (50 mL). The organic layer was washed with water (50 mL), saturated aqueous sodium bicarbonte (50 mL), saturated aqueous sodium chloride (50 mL), dried (Na₂SO₄), filtered and concentrated to afford (S)-5-(chloromethyl)-3,3-dimethyl-tetrahydrofuran-2-one (9.8 g, 75%) as a pale yellow oil.

### Part III—Synthesis of methyl (S)-2,2-dimethyl-3-(oxiran-2-yl)propanoate

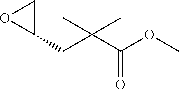

Sodium methoxide solution (25% w/w) (37 mL, 135 mmol) was added to a solution of (S)-5-(chloromethyl)-3,3-dimethyl-tetrahydrofuran-2-one (22.3 g, 137 mmol) in methanol (37 mL). The solution was stirred at room temperature for 2 hours. The reaction mixture was filtered and the filtrate was concentrated. The residue was partitioned between ethyl acetate (100 mL) and water (100 mL). The organic layer was washed with saturated aqueous sodium chloride solution (100 mL), dried (Na₂SO₄), filtered and concentrated. The residue was distilled to afford methyl (S)-2,2-dimethyl-3-(oxiran-2-yl)propanoate (12.3 g, 57%) as a clear, colorless oil (BP=50-53° C. at 1.5 Torr).

### Part IV—Synthesis of N-(3′-(difluoromethoxy)-4,5′-difluoro-[1,1′-biphenyl]-3-yl)-3-(trifluoromethyl)benzenesulfonamide

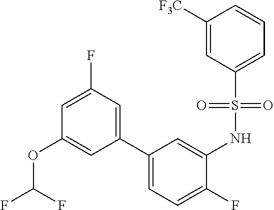 [SMILES: O=S(=O)(Nc1cc(-c2cc(F)cc(OC(F)F)c2)ccc1F)c1cccc(C(F)(F)F)c1]

To a solution of (3-(difluoromethoxy)-5-fluorophenyl)boronic acid (20.0 g, 97.1 mmol), N-(5-bromo-2-fluorophenyl)-3-(trifluoromethyl)benzenesulfonamide (38.7 g, 97.1 mmol), and potassium carbonate (20.1 g, 146 mmol, 1.5 equiv) in 1,4-dioxane (243 ml)/water (60 mL) was added 1,1′-bis (diphenylphosphino)ferrocenepalladium (II) dichloride, toluene (1.6 g, 1.94 mmol, 0.02 equiv). The mixture was heated to 80° C. overnight. Then, the reaction mixture was quenched with water (250 mL) and diluted with tert-butylmethylether (250 mL). The biphasic mixture was filtered through Celite, the phases were separated and the aqueous phase was extracted with tert-butylmethylether (250 mL). The combined organic extracts were washed with saturated aqueous sodium chloride (250 mL). The organic extracts were treated with charcoal, filtered through Celite, and rinsed with tert-butylmethylether. The filtrates were concentrated and then the residue was dissolved in tert-butylmethylether (100 mL) and then diluted with hexane (500 mL). The resulting mixture was stirred at room temperature for 2 hours and the resulting suspension was diluted with hexane (200 mL), filtered, rinsed with hexane (100 mL) and dried to afford N-(3′-(difluoromethoxy)-4,5′-difluoro-[1,1′-biphenyl]-3-yl)-3-(trifluoromethyl)benzenesulfonamide (31.5 g, 68%).

### Part V—Synthesis of (S)—N-(3′-(difluoromethoxy)-4,5′-difluoro-[1,1′-biphenyl]-3-yl)-N-((4,4-dimethyl-5-oxotetrahydrofuran-2-yl)methyl)-3-(trifluoromethyl)benzenesulfonamide

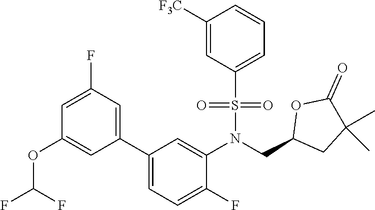 [SMILES: CC1(C)C[C@@H](CN(c2cc(-c3cc(F)cc(OC(F)F)c3)ccc2F)S(=O)(=O)c2cccc(C(F)(F)F)c2)OC1=O]

A mixture of methyl (S)-2,2-dimethyl-3-(oxiran-2-yl)propanoate (11.1 g, 70.5 mmol), of N-(3′-(difluoromethoxy)-4,5′-difluoro-[1,1′-biphenyl]-3-yl)-3-(trifluoromethyl)benzenesulfonamide (26.0 g, 54.2 mmol), tetra-n-butylammonium bromide (1.75 g, 5.42 mmol), and potassium carbonate (750 mg, 5.42 mmol) was stirred at 90° C. overnight. Then, the reaction mixture was quenched with saturated aqueous sodium chloride (250 mL), and extracted with ethyl acetate (250 mL). The organic extracts were washed with saturated aqueous sodium chloride (250 mL), dried (Na₂SO₄), filtered and concentrated. The resulting residue was dissolved in methanol (150 mL) and warmed to 40° C. for fifteen minutes. The mixture was allowed to cool to room temperature and then further cooled to 0° C. and stirred for thirty minutes. The suspension was filtered, rinsed with methanol (30 mL), and dried to afford (S)—N-(3′-(difluoromethoxy)-4,5′-difluoro-[1,1′-biphenyl]-3-yl)-N-((4,4-dimethyl-5-oxotetrahydrofuran-2-yl)methyl)-3-(trifluoromethyl)benzenesulfonamide (16.0 g, 49%).

### Part VI—Synthesis of sodium (S)-3-(6-(3-(difluoromethoxy)-5-fluorophenyl)-4-((3-(trifluoromethyl)phenyl)sulfonyl)-3,4-dihydro-2H-benzo[b][1,4]oxazin-2-yl)-2,2-dimethylpropanoate

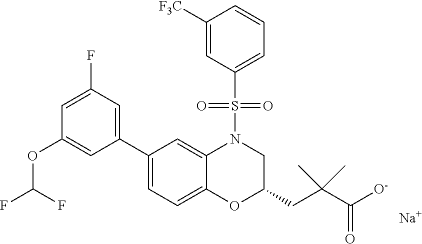 [SMILES: CC(C)(C[C@H]1CN(S(=O)(=O)c2cccc(C(F)(F)F)c2)c2cc(-c3cc(F)cc(OC(F)F)c3)ccc2O1)C(=O)[O-] ; [Na+]]

A solution of (S)—N-(3′-(difluoromethoxy)-4,5′-difluoro-[1,1′-biphenyl]-3-yl)-N-((4,4-dimethyl-5-oxotetrahydrofuran-2-yl)methyl)-3-(trifluoromethyl)benzenesulfonamide (23.6 g, 39.0 mmol), tetra-n-butylammonium bromide (2.26 g, 3.90 mmol), and sodium hydroxide (6.24 g, 156 mmol) in THF (39 mL) was heated to 65° C. for 23 hours. Then, the reaction mixture was quenched with water, extracted with ethyl acetate, dried (Na₂SO₄), filtered and concentrated. The resulting residue was dissolved in isopropyl alcohol (23 mL), and stirred until solids developed. The thick suspension was diluted with isopropyl alcohol (46 mL) and stirred at room temperature overnight. Next, the suspension was filtered, rinsed with isopropyl alcohol (46 mL), and dried to afford sodium (S)-3-(6-(3-(difluoromethoxy)-5-fluorophenyl)-4-((3-(trifluoromethyl)phenyl)sulfonyl)-3,4-dihydro-2H-benzo[b][1,4]oxazin-2-yl)-2,2-dimethylpropanoate (18.3 g, 78%). ¹H-NMR (400 MHz, CD₃OD) δ 8.18 (d, J=8.0 Hz, 1H), 8.07 (d, J=8.0 Hz, 1H), 7.95 (d, J=2 Hz, 1H), 7.89-7.85 (m, 2H), 7.47 (dd, J=8.4 Hz, 2.4 Hz, 1H), 7.41 (t, J=74 Hz, 1H), 7.31 (m, 1H), 7.22 (m, 1H), 7.12 (m, 1H), 6.89 (d, J=8.8 Hz, 1H), 4.61 (dd, 1H), 3.58 (m, 1H), 3.23 (m, 1H), 1.82 (dd, 1H), 1.43 (dd, 1H), 0.99 (s, 3H), 0.90 (s, 3H). ¹³C-NMR (400 MHz, CD₃OD) δ 180.7, 163.9 (d, J=245 Hz), 152.4 (dt, J=12.4, 3.4 Hz), 147.6, 143.0 (d, J=9.6 Hz), 138.8, 131.8, 131.6, 130.4 (q, J=3.3 Hz), 129.9 (q, J=32.9 Hz), 130.2 (d, 2.3 Hz), 123.0 (q, J=273 Hz), 125.1, 123.5 (q, J=3.7 Hz), 123.1, 121.7, 116.2 (t, J=258 Hz), 118.2, 112.2 (d, J=2.5 Hz), 109.6 (d, J=22.4 Hz), 104.8 (d, J=25.7 Hz), 71.4, 48.8, 43.7, 40.7, 28.5, 25.4. ¹⁹F-NMR (400 MHz, CD₃OD) δ −61.2, −82.6 (d, J=73.6 Hz), −109.5 (t, dd, J=9.7 Hz, 9.7 Hz). (ES, m/z): (M−H)⁻ 602.

### Example 2—Biological Assays for Agonist Activity Towards RORγ

The following compound (hereinafter “Compound A”) was tested for ability to increase RORγ activity using (i) a RORγ-Ligand Binding Domain (LBD) TR-FRET Assay, and (ii) a Gal4-RORγ Luciferase Reporter Assay in HEK-293T Cells:

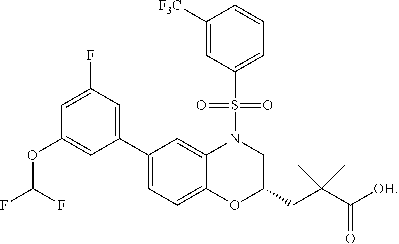 [SMILES: CC(C)(C[C@H]1CN(S(=O)(=O)c2cccc(C(F)(F)F)c2)c2cc(-c3cc(F)cc(OC(F)F)c3)ccc2O1)C(=O)O]

Assay procedures and results are described below.

### Part I—Procedures for RORγ-Ligand Binding Domain TR-FRET Assay

HIS-tagged RORγ-LBD protein was expressed in SF9 cells using a baculovirus expression system. The lysate was diluted in assay buffer (50 mM Tris pH 7.0, 50 mM KCl, 1 mM EDTA, 0.1 mM DTT, 0.01% BSA) to obtain RORγ-LBD final concentration of −3 nM in a 384-well assay plate (need to titrate for each batch of protein).

A stock of biotinylated-LXXLL peptide from coactivator SRC1 (Biotin-CPSSHSSLTERHKILHRLLQEGSPS) was prepared in assay buffer and added to each well (200 nM final concentration). A solution of Europium tagged anti-HIS antibody (0.6 nM final concentration) and APC-conjugated streptavidin (30 nM final concentration) were also added to each well. RORγ antagonist ursolic acid was also included at a final concentration of 2 μM. Compounds were diluted in DMSO and further diluted in assay buffer with a final DMSO concentration at 1%. The highest concentration of test compound analyzed was 10 μM.

The final assay mixture was incubated overnight at 4° C. or 2 hours at room temperature, and the fluorescence signal was measured on an Envision plate reader: (Excitation filter=340 nm; APC emission=665 nm; Europium emission=615 nm; dichroic mirror=D400/D630; delay time=100 μs, integration time=200 μs). 50% Effective concentration (EC₅₀) values for test compounds were calculated from the quotient of the fluorescence signal at 665 nm divided by the fluorescence signal at 615 nm. The quotient of the fluorescence signals in the absence of ursolic acid or test compound is set as 100. Max Response is defined as the upper plateau in the signal as determined by line-fit using a 4-parameter logistic model in PRISM (GraphPad).

### Part II—Procedures for Gal4-RORγ Luciferase Reporter Assay in HEK-293T Cells

### Transfection of HEK-293 Cells

In the following protocol, HEK-293 cells were transfected with a construct comprising the Gal4 DNA binding domain fused to the ligand binding domain of RORγ (Gal4-RORγ-LBD) in a pcDNA3.1neo plasmid, and also with a reporter construct comprising pGL4.31 Gal4-luciferase (Promega). Control cells were prepared similarly using empty pcDNA3.1neo and pGL4.31 vectors.

Trans-IT reagent (Minis, 60 μL) at room temperature was added drop wise to OptiMEM (Invitrogen, 1.5 ml). This reagent mixture was mixed by inversion then incubated for 5 to 30 minutes at room temperature. It then was added to a solution of both expression vectors (5 μg each), mixed, and incubated at room temperature for about 20 minutes. HEK-293 cells were harvested from incubation flasks by removing the media, treating with TrypLE Express (Invitrogen), and incubating until the cells detached from the bottom of the flask (approximately 2-5 minutes). 10 Million cells were collected by centrifugation and re-suspended in 10 mL of Dulbecco's Modified Eagle Medium, High Glucose (DMEM, Invitrogen) containing 10% Fetal Bovine Serum and 100 IU each of penicillin and streptomycin. The re-suspended cells and the transfection mixture were added to a T75 flask, mixed and incubated overnight at 37° C. and 5% CO₂.

### Assay for RORγ Activity

The cells were harvested as described above, counted, and centrifuged to obtain the desired number of cells, then re-suspended in complete growth media at 0.75×10⁶ cells/mL. The RORγ antagonist, ursolic acid, was added to the cells at a final concentration of 2 μM. Cells were plated at 20 μL of cell suspension/well (10,000-15,000 cells/well) in white tissue culture treated 384 well plates. Test compounds were dissolved at 10 mM in DMSO then diluted into complete growth medium to 5× the final intended test concentration. These drug stock solutions, 5 μL/well were added to the tissue culture plate. The final DMSO concentration was 0.2%. The plates were briefly centrifuged then incubated overnight at 37° C. and 5% CO₂. To conduct the assay, the tissue culture plates were allowed to equilibrate to room temperature and One-Glo luciferase reagent (Promega, 25 μL/well) was added. The plates were briefly centrifuged then incubated at room temperature for 10 minutes. The luciferase intensity was read on an Envision plate reader (Perkin Elmer). RORγ activity was determined relative to controls and plotted as a function of test compound concentration using PRISM (GraphPad) to determine a 50% effective concentration (EC₅₀). The luciferase signal in the absence of ursolic acid or test compound is defined at 100. The Max Response is the upper plateau in the signal as determined by line-fit using a 4-parameter logistic model in PRISM (GraphPad).

### Part III—Results

Experimental results are provided in Table 1 below. The symbol “++++” indicates an EC₅₀ less than 0.5 μM. The symbol “+++” indicates an EC₅₀ in the range of 0.5 μM to 5 μM. The symbol “++” indicates an EC₅₀ in the range of greater than 5 μM to 10 μM. The symbol “+” indicates an EC₅₀ greater than 10 μM. The symbol “****” indicates a value greater than 200. The symbol “***” indicates a value in the range of greater than 150 to 200. The symbol “**” indicates a value in the range of greater than 90 to 150. The symbol “*” indicates a value in the range of 25 to 90.

TABLE 1

| | |
|---|---|
| TR-FRET Assay | Gal4-RORγ Assay |

| | | Max | | Max |
|---|---|---|---|---|
| Compound No. | EC₅₀ | Response | EC₅₀ | Response |
| A | ++++ | **** | ++++ | ** |

### Example 3—Administration of Compound A to Human Patient Suffering from Bladder Cancer

A human, female patient suffering from bladder cancer was orally administered a 450 mg dose of Compound A (structure shown below) two times per day for two months and then subjected to computerized tomography (CT) imaging to evaluate change in her bladder cancer. Imaging results showed that there was a 24% reduction in bladder cancer burden compared to bladder cancer burden present at the start of treatment using Compound A. Compound A has the following formula:

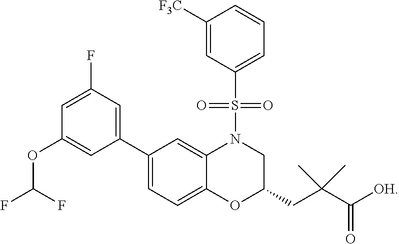 [SMILES: CC(C)(C[C@H]1CN(S(=O)(=O)c2cccc(C(F)(F)F)c2)c2cc(-c3cc(F)cc(OC(F)F)c3)ccc2O1)C(=O)O]

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating a cancer that is a squamous cell carcinoma located in a patient's head and/or neck, comprising administering to a patient in need thereof a therapeutically effective amount of a compound of Formula I, to thereby treat the cancer, wherein Formula I is represented by: 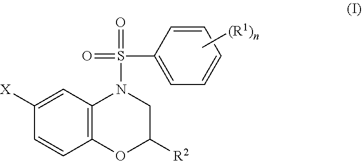
or a pharmaceutically acceptable salt thereof; wherein:
X is phenyl substituted by 1, 2, or 3 substituents independently selected from the group consisting of C₁₋₂ fluoroalkoxy, C₁₋₂ alkoxy, chloro, fluoro, and C₁₋₂ fluoroalkyl;
R¹ represents independently for each occurrence C₁₋₂ fluoroalkyl, chloro, fluoro, cyclopropyl, or C₁₋₃ alkyl;
R² is —(C₁₋₆ alkylene)—CO₂H; and
n is 1 or 2.

2. The method of claim 1, wherein the squamous cell carcinoma is located in the patient's head.

3. The method of claim 1, wherein the squamous cell carcinoma is located in the patient's neck.

4. The method of claim 1, wherein the squamous cell carcinoma is located in the patient's head and neck.

5. The method of claim 1, wherein the squamous cell carcinoma is located in the patient's mouth or throat.

6. The method of claim 1, wherein the squamous cell carcinoma is located in the patient's throat.

7. The method of claim 1, wherein the squamous cell carcinoma is located in the patient's nasal cavity or nasopharynx.

8. A method of treating a cancer that is a urothelial carcinoma, comprising administering to a patient in need thereof a therapeutically effective amount of a compound of Formula I, to thereby treat the cancer, wherein Formula I is represented by: 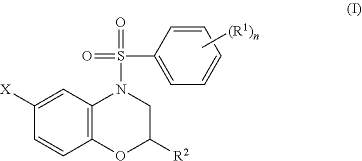
or a pharmaceutically acceptable salt thereof; wherein:
X is phenyl substituted by 1, 2, or 3 substituents independently selected from the group consisting of C₁₋₂ fluoroalkoxy, C₁₋₂ alkoxy, chloro, fluoro, and C₁₋₂ fluoroalkyl;
R¹ represents independently for each occurrence C₁₋₂ fluoroalkyl, chloro, fluoro, cyclopropyl, or C₁₋₃ alkyl;
R² is —(C₁₋₆ alkylene)—CO₂H; and
n is 1 or 2.

9. (canceled)

10. (canceled)

11. (canceled)

12. The method of claim 1, wherein X is phenyl substituted by —OC(H)F₂ and fluoro.

13. (canceled)

14. (canceled)

15. (canceled)

16. (canceled)

17. The method of claim 1, wherein the compound is 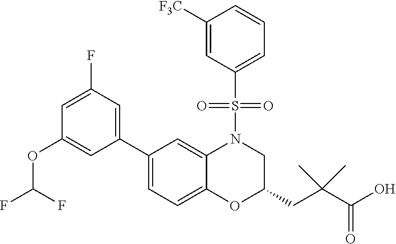 [SMILES: CC(C)(C[C@H]1CN(S(=O)(=O)c2cccc(C(F)(F)F)c2)c2cc(-c3cc(F)cc(OC(F)F)c3)ccc2O1)C(=O)O]
or a pharmaceutically acceptable salt thereof.

18. The method of claim 1, wherein the compound is a pharmaceutically acceptable salt of

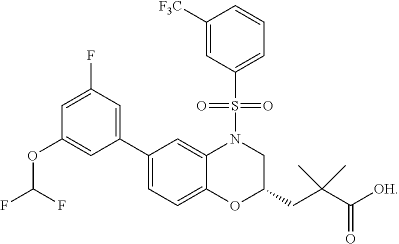 [SMILES: CC(C)(C[C@H]1CN(S(=O)(=O)c2cccc(C(F)(F)F)c2)c2cc(-c3cc(F)cc(OC(F)F)c3)ccc2O1)C(=O)O]

19. The method of claim 1, wherein the compound is

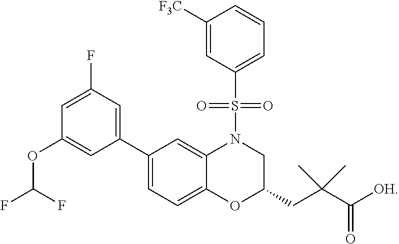 [SMILES: CC(C)(C[C@H]1CN(S(=O)(=O)c2cccc(C(F)(F)F)c2)c2cc(-c3cc(F)cc(OC(F)F)c3)ccc2O1)C(=O)O]

20. The method of claim 1, wherein the compound is administered orally to the patient.

21-43. (canceled)

44. The method of claim 1, wherein a unit dosage of the compound is orally administered to the patient twice per day, the unit dosage contains from about 400 mg to about 500 mg of the compound, and wherein if the patient experiences an adverse event due to the compound, then thereafter for a period of at least 2 days the compound is orally administered to the patient at a reduced unit dose two times per day, wherein said reduced unit dose contains from about 300 mg to about 400 mg of the compound.

45. (canceled)

46. (canceled)

47. The method of claim 1, wherein a unit dosage of the compound is orally administered to the patient twice per day, the unit dosage contains from about 400 mg to about 500 mg of the compound, and wherein if the patient experiences an adverse event due to the compound, then thereafter for a period of at least 2 days the compound is orally administered to the patient at a reduced unit dose two times per day, wherein said reduced unit dose contains about 350 mg of the compound

48. (canceled)

49. The method of claim 1, wherein a unit dosage of the compound is orally administered to the patient twice per day, the unit dosage contains from about 400 mg to about 500 mg of the compound, and wherein if the patient experiences an adverse event due to the compound, then thereafter for a period of at least 2 days the compound is orally administered to the patient at a reduced unit dose two times per day, wherein said reduced unit dose contains about 300 mg of the compound.

50-55. (canceled)

56. The method of claim 44, wherein the adverse event is gastric distress.

57. The method of claim 44, wherein the adverse event is diarrhea

58. The method of claim 44, wherein the adverse event is one or more of fatigue, anemia, poor appetite, or dry mouth.

59-64. (canceled)

65. The method of claim 1, wherein the patient is an adult human.

66-67. (canceled)
